(19) United States
(12) Patent Application Publication
(10) Pub. No.: US 2016/0174885 A1
(43) Pub. Date: Jun. 23, 2016

(54) MICRO-NEEDLE AND SENSOR FOR DETECTING NITROGEN MONOOXIDE COMPRISING THE SAME

(71) Applicant: POSTECH ACADEMY-INDUSTRY FOUNDATION, Gyeongsangbuk-do (KR)

(72) Inventors: Sei Kwang HAHN, Gyeongsangbuk-do (KR); Dohee KEUM, Busan (KR); Ho Sang JUNG, Seoul (KR); Hyemin KIM, Daegu (KR); Myeong Hwan SHIN, Gyeongsangbuk-do (KR)

(21) Appl. No.: 14/855,727

(22) Filed: Sep. 16, 2015

(30) Foreign Application Priority Data
Sep. 16, 2014 (KR) 10-2014-0122889

(51) Int. Cl. A61B 5/145; A61B 5/1473

(52) U.S. Cl. CPC A61B 5/14542; A61B 5/1473

(57) ABSTRACT

The present invention relates to a microneedle, a sensor for detecting nitrogen monoxide, including the microneedle, a medical apparatus including the microneedle, and a manufacturing method thereof. The microneedle of the present invention may detect whether nitrogen monoxide is present or not by using electrochemical principles. Further, a change in concentration of nitrogen monoxide may be sensed in real time. The effects of detecting nitrogen monoxide may be used to diagnose cancer and forecast the size and growth degree of a tumor.

### TECHNICAL FIELD

The present invention relates to a microneedle and a sensor for detecting nitrogen monoxide, including the same.

### BACKGROUND ART

In general, a microneedle is used for delivery of active materials such as a drug and a vaccine in vivo, detection of an analyte in vivo, and biopsy. As a method of using a microneedle, a form in which a certain number of holes are formed on the skin by using a microneedle device such as a roller to which the microneedle is attached, and then a drug is applied thereon, a form in which the surface of a microneedle is coated with an active ingredient (effective ingredient) to allow the active ingredient to be administered simultaneously with perforation of the skin, a form in which when injected with a microneedle using a polymer (a biodegradable polymer or a water-soluble polymer), an active material included in the microneedle is decomposed or dissolved in the skin and diffused, and the like are generally used.

Meanwhile, it is most important to early diagnose cancer which is characterized by metastasis, and an endoscope is used as the most common and easiest method in order to prevent cancer. Various kinds of endoscopes for diagnosing cancer more accurately have been abundantly developed, and representative examples thereof include an electronic endoscope, a capsule endoscope, a 3D endoscope, and the like. After cancer is primarily classified by using images obtained from an endoscope using a dye staining method, the tissue of interest is collected, and then it is determined whether cancer is malignant or not. However, for the current method, it is essential to perform a tissue examination in order to accurately determine whether the tumor is malignant or not, and there are problems in that it takes a long time to determine whether the tumor is malignant through the tissue examination, and images obtained through the staining method of the endoscope may be sometimes incorrectly analyzed, thereby leading to the occurrence of misdiagnosis.

The present invention has been made in an effort to solve the aforementioned problems and provide a sensor which is capable of diagnosing cancer by a non-invasive method within a short period of time by using a microneedle.

### CITATION LIST

### Non-Patent Document

(Non-Patent Document 1) Microneedle Electrodes Toward an Amperometric Glucose Sensing Smart Patch, Michael A et al., Advanced Healthcare Materials, 2013
(Non-Patent Document 2) Real-time Electrical Detection of Nitric Oxide in Biological Systems with Sub-Nanomolar Sensitivity, Shan Jiang et al., Nature Communications, 2013

### SUMMARY OF THE INVENTION

The present invention relates to a microneedle, a sensor for detecting nitrogen monoxide, including the microneedle, a medical apparatus including the microneedle, and a manufacturing method thereof.

The present invention provides a microneedle in which a microneedle base; an adhesive polymer layer; a conductive polymer layer; and a nitrogen monoxide bonding molecule layer including iron ions are sequentially stacked.

The present invention also provides a sensor for detecting nitrogen monoxide, the sensor including: the microneedle; and an electrode.

The present invention also provides a sensor for diagnosing cancer, the sensor including: the microneedle; and an electrode.

The present invention also provides an endoscope including the microneedle, the sensor for detecting nitrogen monoxide, or the sensor for diagnosing cancer.

The present invention also provides a method for manufacturing a microneedle, the method including: forming an adhesive polymer layer on a microneedle base by mixing the microneedle base with an adhesive polymer; forming a conductive polymer layer on the adhesive polymer layer through a solution process by bringing the adhesive polymer layer into contact with a conductive polymer solution; and forming a nitrogen monoxide bonding layer on the conductive polymer layer by bringing the conductive polymer layer into contact with a nitrogen monoxide bonding molecule layer including iron ions.

The microneedle of the present invention may detect whether nitrogen monoxide is present or not by using electrochemical principles. Further, a change in concentration of nitrogen monoxide may be sensed in real time. The effects of detecting nitrogen monoxide may be used to diagnose cancer and forecast the size and growth degree of tumor.

### BRIEF DESCRIPTION OF THE DRAWINGS

FIG. 1 is a cross-sectional view of a sensor including a microneedle according to an exemplary embodiment of the present invention. In the drawing, reference numeral 1 denotes a PEDOT channel coated on the surface of a microneedle base, reference numeral 2 denotes a layer composed of gold as an electrode for connecting a working electrode and a counter electrode to both ends thereof, and reference numeral 3 denotes a layer of titanium and chromium, which may adhere the microneedle below the gold layer to gold. Reference numeral 4 denotes an electrode composed of titanium for connecting a reference electrode, and reference numeral 5 is a microneedle panel composed of polycaprolactone. One end of the electrode shown by reference numerals 2 and 3 is connected to a working electrode and the other end thereof is connected to a counter electrode, and reference numeral 4 is connected to the reference electrode.

FIG. 2A illustrates an example to which a sensor including the microneedle of the present invention is applied. Reference numeral 6 denotes a copper conducting wire which connects the working electrode to an electrode at one end of the microneedle, and the electrode at the end of the microneedle is soldered in order to electrically connect the electrode to a probe station. The counter electrode is also connected to the electrode on the opposite side by identically connecting the copper conducting wire to the electrode. Reference numeral 7 denotes an orange portion that covers the electrode and edge portion of the microneedle, illustrating that the portion other than the channel of the microneedle is coated with a polymer and waterproofed. Reference numeral 8 illustrates that the reference electrode is connected.

FIG. 2B is an exemplary embodiment of the present invention, illustrating that a sensor including the microneedle according to the present invention is applied to the skin of a mouse.

FIG. 2C is an exemplary embodiment of the present invention, illustrating that a sensor including the microneedle according to the present invention is applied to an endoscope. Reference numeral 8 denotes an endoscope part which captures white light, reference numeral 10 denotes an endoscope part which captures fluorescence, and reference numeral 11 denotes an endoscope part to be equipped with the sensor.

FIGS. 3A, 3B, 3C and 3D illustrate a result of observing the surface of the microneedle according to the coating at each step by using an optical microscope (FIG. 3A: manufacturing a microneedle base composed of polycaprolactone; FIG. 3B: coating PEDOT by processing the PEDOT with a solution without dopamine coating; FIG. 3C: coating polycaprolactone with dopamine; and FIG. 3D: coating the PEDOT by processing the PEDOT with a solution after dopamine coating).

FIGS. 4A, 4B and 4C illustrate a result of observing the surface of the microneedle according to the coating at each step by using a scanning electron microscope. FIGS. 4A, 4B, and 4C illustrate a side view (left) of the microneedle and an image (right) of the end of the microneedle according to the surface coating (FIG. 4A: the initial surface of the microneedle base formed of polycaprolactone, illustrating that the surface is clean; FIG. 4B: illustrates that the surface was not smoothly coated when the surface is coated with a PEDOT polymer without dopamine; and FIG. 4C: the surface obtained by being coated with the PEDOT after being coated with dopamine, illustrating that the surface is coated well).

FIG. 5 is a graph confirming iron of hemin molecules present on the surface of the microneedle sensor channel by using an X-ray spectrometer (energy dispersion X-ray, EDX) attached to a scanning electron microscope.

FIG. 6 is a graph quantitatively confirming the content of iron of hemin molecules present on the microneedle channel by using a photoelectron spectrometer (X-ray photoelectron spectroscopy (XPS).

FIGS. 7A and 7B illustrate the confirmation of mechanical properties of the microneedle, in which FIG. 7A illustrates a result of deriving failure stress by measuring the intensity of force depending on the compression of the microneedle, and FIG. 7B illustrates a pressure graph over the displacement obtained from the result of FIG. 7B.

FIGS. 8A, 8B, and 8C illustrate observation of the skin of a mouse to which the microneedle is applied, in which FIG. 8A is a side view of the skin of the mouse, FIG. 8B is a cross-sectional view of the skin of the mouse, and FIG. 8C is a side view in which the skin of the mouse is cryosectioned.

FIG. 9 is a graph illustrating that the surface of a sensor including the microneedle according to the coating at each step is observed by a circulating current method.

FIG. 10 is a voltage-current graph according to the scan rate during the measurement by a circulating current method and a graph illustrating that the movement of the oxidation-reduction peaks of a hemin group is observed.

FIG. 11 is a circulating current graph obtained by adding nitrogen monoxide to the electrolyte at each concentration.

FIG. 12 illustrates a result of measuring the resistance value of the surface of a sensor including the microneedle by an electrochemical method.

FIG. 13 is a scanning electron microscope image in which the surface before and after the end of the microneedle is applied to the skin of a mouse is observed at 50-, 1,000-, 3,000-, and 10,000-fold magnifications from the top.

FIG. 14 is a circulating current graph of the microneedle used after a detection test is finished.

FIG. 15 is a circulating current graph of the microneedle of which measurement is performed 50 consecutive times.

FIG. 16 illustrates a result of measuring the cell viability by the sensor including the microneedle.

FIG. 17 illustrates a result of observing a change in current value by flowing an aqueous solution including nitrogen monoxide in the sensor including the microneedle at each concentration.

FIG. 18 illustrates a result of analyzing changes in current of the microneedle for various molecules and polysaccharides (galactose, glucose, iron ions, peroxide, lysozyme, and albumin) which may affect the detection of the sensor.

FIG. 19 illustrates a result of measuring the capability of detecting nitrogen monoxide dissolved in a cell culture solution in which various materials are present.

FIG. 20 illustrates a result of quantifying the absorbance at the wavelength of 540 depending on the concentration of nitrogen monoxide by using a grease reagent.

FIG. 21A is a graph in which the amount of nitrogen monoxide generated from the raw 246.7 macrophage cells depending on the treatment concentration of the lipopolysaccharide body is measured.

FIG. 21B is a graph in which the amount of nitrogen monoxide depending on the concentration of aminoguanidine was measured when the raw 246.7 macrophage cells are treated simultaneously with a lipopolysaccharide body and aminoguanidine.

FIG. 22 illustrates a result of measuring nitrogen monoxide, which is released from normal cells, cells treated with a lipopolysaccharide, and cells treated simultaneously with a lipopolysaccharide and even aminoguanidine, through the sensor.

FIG. 23 is a graph in which the amount of nitrogen monoxide substantially released from the cells is converted by using the current change value measured from the sensor including the microneedle.

FIG. 24 illustrates that the skin tissue of a mouse with skin cancer was observed, and that cancer is detected by applying the sensor including the microneedle to the skin tissue of the mouse.

FIG. 25 illustrates the current values obtained by inserting the microneedle into the skin of a normal mouse and the skin around the cancer cells of the mouse with skin cancer.

FIG. 26 illustrates the current values obtained by alternately inserting the microneedle into the skin of a normal mouse and the skin of the mouse with skin cancer.

### DETAILED DESCRIPTION

The present invention provides a microneedle in which a microneedle base; an adhesive polymer layer; a conductive polymer layer; and a nitrogen monoxide bonding molecule layer including iron ions are sequentially stacked.

The present invention also provides a method for manufacturing a microneedle, the method including: forming an adhesive polymer layer on a microneedle base by mixing the microneedle base with an adhesive polymer; forming a conductive polymer layer on the adhesive polymer layer through a solution process by bringing the adhesive polymer layer into contact with a conductive polymer solution; and forming a nitrogen monoxide bonding layer on the conductive polymer layer by bringing the conductive polymer layer into contact with a nitrogen monoxide bonding molecule layer including iron ions.

The microneedle of the present invention is comprised of a microneedle base, an adhesive polymer layer, a conductive polymer layer, and a nitrogen monoxide bonding molecule layer. The conductive polymer layer and the nitrogen monoxide bonding molecule layer are for detecting nitrogen monoxide, and the adhesive polymer layer is necessary for stably constituting the conductive polymer layer and the nitrogen monoxide bonding molecule layer.

In the present specification, the microneedle base forms a basic framework of a microneedle, exhibiting the shape of the microneedle. The microneedle of the present invention is completed by forming additional coating layers on the microneedle base. The microneedle base may be manufactured by a common publicly known method according to the constituent material. For example, a microneedle base composed of a biodegradable polymer may be formed by putting the biodegradable polymer into a mold for a microneedle, adding heat to the mold, and cooling the mold. In the case of a microneedle base composed of aluminum oxide, aluminum nickel, nickel oxide or stainless steel and the like, a microneedle may be formed by using an etching method, and in the case of a microneedle base composed of stainless steel, a microneedle may be formed by an etching method or a metal mold casting method.

In one specific exemplary embodiment, the microneedle base may be one or more selected from the group consisting of polyvinyl alcohol, polyethylene glycol, polylactide, polyglycolide, polyethylene oxide, polydioxanone, polyphosphazene, polyanhydride, polyamino acid, polyacrylate, polyacrylamide, polyurethane, polysiloxane, polyvinylpyrrolidone, polycaprolactone, polymethylmethacrylate, polyethylene, polyamide, polydimethylsiloxanes, polyester, polyorthoester, polycyanoacrylates, polyphosphazenes, polyvinylchrolide, polymethylpentene, polynitrobenzyl, polyaminoester, cellulose acetate butyrate, cellulose triacetate, polyethylene terephthalate, Teflon (polytetrafluoroethylene), stainless steel, silicon, silicon oxide, aluminum, aluminum oxide, nickel oxide, and SU-8 (an epoxy-based negative type photoresist), but is not limited thereto. In one specific exemplary embodiment, the microneedle base may be composed of a biodegradable polymer, and the biodegradable polymer may be a hydrophobic polymer. In one specific exemplary embodiment, polycaprolactone (PCL) may be used as a base of the microneedle, and polycaprolactone is a biodegradable material and has very high biostability. Further, strength of the microneedle may be appropriately adjusted by adjusting the molecular weight of polycaprolactone. In one specific exemplary embodiment, the molecular weight of polycaprolactone may be 4,000 to 10,000 kDa, 5,000 to 9,000 kDa, 6,000 to 85,000 kDa or 7,000 to 8,300 kDa, but is not limited thereto.

An adhesive polymer layer may be formed on the microneedle base by mixing the microneedle base with an adhesive polymer. In one specific exemplary embodiment, the adhesive polymer may be chitosan, silk, collagen, fibronectin, vitronectin, rubber, or polydopamine, but is not limited thereto. For example, the adhesive polymer may include a catechol group, and may be polydopamine. Dopamine is a polymer having adhesive properties and high biostability and causes self-polymerization on the hydrophobic surface of a polycaprolactone microneedle according to the oxidation reaction, so that a dopamine polymer is grown on the surface of the microneedle base composed of polycaprolactone, thereby allowing the surface of the microneedle base to be coated. Accordingly, the surface of the microneedle exhibits hydrophilicity, and is simultaneously modified into a surface having very high adhesion. Such a surface modification facilitates coating of PEDOT to be subsequently used, and adhesion is excellent, and thus stability of the microneedle is improved.

In one specific exemplary embodiment, subjecting the microneedle base to UV treatment or ozone plasma treatment may be additionally included before forming the adhesive polymer layer on the microneedle base. The UV treatment or the ozone plasma treatment is a surface modification method publicly known in the art, and the surface coating capability of a conductive polymer layer to be added may be improved through the treatment. For example, the UV treatment may be performed by using a UVO cleaner apparatus to perform the UV for 25 to 35 minutes or 30 minutes, the ozone plasma treatment may be performed by using a reactive ion etching (RIE) apparatus (SNTEK BSC5004) to treat the ozone plasma under a vacuum of 5×10⁻⁶ torr or less under the oxygen atmosphere at a power of 100 for 50 to 70 seconds or 60 seconds.

In one specific exemplary embodiment, the conductive polymer may be polyacetylene, polyaniline, polypyrrole, polythiophene, poly(1,4-phenylenevinylene), poly(1,4-phenylene sulfide), poly(fluorenylene ethynylene), polyisothianaphthene, polythienylene vinylene, polyphenylene vinylene, polyphenylene sulfide, polyhexylthiophene, PEDOT, or derivatives thereof, but is not limited thereto. Poly(3,4-ethylenedioxythiophene) (PEDOT) is a bluish polymer, and a conductive polymer which is innocuous in vivo, has good biostability, and is easy to be handled in the process. Due to these characteristics, the PEDOT is used for a channel or an electrode of a sensor in various fields such as a biosensor, a semiconductor, and a solar cell. The chains of a PEDOT polymer have a number of pi electrons, form a pi-pi bond with pi electrons which other molecules have, and thus affect the conductivity of the PEDOT polymer when doped with an n-type or p-type dopant. The PEDOT is present in a solution state, mixed with a surfactant PSS in an aqueous solution, and in order to use a polymer having a molecular weight within a suitable range, the PEDOT is filtered by a filter, and then coated on the microneedle coated with dopamine, which becomes hydrophilic, through a solution process.

In one specific exemplary embodiment, the solution process may be performed by immersing a microneedle base, on which an adhesive polymer layer is formed, in a conductive polymer solution, and drying the microneedle base.

In one specific exemplary embodiment, a nitrogen monoxide bonding molecule including iron ions may be a porphyrin ring or a hemin molecule, which has pi electrons in the core thereof. The hemin molecule having a structure similar to hemoglobin is an unpaired orbital material having trivalent iron ions in the core, and rapidly captures nitrogen monoxide in vivo to cause a nitrosylation. Extra pi electrons present in the center of the porphyrin ring of the hemin molecule are bonded to other pi electrons to form a pi-pi bond, and are bonded to a number of extra pi electrons present in the PEDOT polymer chains, thereby forming a channel of the sensor. When the PEDOT polymer is used as a channel of the sensor, a small amount of the PEDOT polymer is decomposed in the air due to the hydrophilic tendency which is an inherent property of poly(3,4-ethylenedioxythiophene) polystyrene sulfonate 1000 (PEDOT: PSS 1000) which is used for coating, but when one layer of a hydrophobic molecule hemin is coated on the PEDOT layer, the channel is prevented from being damaged and stability of the microneedle is increased by entirely covering a PEDOT channel.

The microneedle may be formed on a microneedle pad. The microneedle pad means a plate on which the microneedle is formed, and the function of the sensor may be imparted to the microneedle by depositing an electrode on the microneedle pad. For example, for the microneedle of the present invention, 10 needles in both width and length directions are present in a square form on the microneedle pad, and thus, a total of 100 needles may be formed.

Accordingly, the present invention also provides a sensor for detecting nitrogen monoxide, including the microneedle and an electrode. The present invention also provides a method for manufacturing a sensor for detecting nitrogen monoxide, the method including depositing an electrode on the microneedle pad in which the microneedle is formed.

In one specific exemplary embodiment, the electrode may be composed of one or more selected from the group consisting of nickel, chromium, titanium, gold, silver, and platinum. Nickel, chromium, titanium and the like may be used as an electrode for being adhered to a gold or silver electrode.

In one specific exemplary embodiment, the electrode may include a reference electrode, a working electrode, and a counter electrode.

The sensor for detecting nitrogen monoxide according to the present invention may diagnose cancer by detecting the amount of nitrogen monoxide around the cancer cell. Accordingly, the present invention provides a sensor for diagnosing cancer, including the microneedle and the electrode.

In most cancers such as esophageal, gastric, colorectal, and skin cancers, an inducible nitric oxide synthase gene is excessively expressed for the process of metastasis and apoptosis of cancer cells, and a large amount of nitrogen monoxide is secreted from the cancer cells by the inducible nitric oxide synthase gene. Nitrogen monoxide is always secreted from the tissues around the cancer cell by 1,000 times or more than from the normal cells, and thus, the concentration of nitrogen monoxide is maintained at a high concentration at a micromole level for several days. Nitrogen monoxide is a radical molecule in a very unstable state, and is rapidly diffused along the blood vessels, the lymph vessels, and tissues when generated in vivo. When nitrogen monoxide combines with hemoglobin in the blood in the in vivo environment, nitrogen monoxide is captured by the iron ions of the hemoglobin, and annihilated.

That is, the amount of nitrogen monoxide present around the cancer cells is an important biomarker which may diagnose cancer. The sensor including the microneedle according to the present invention may measure the amount of nitrogen monoxide, which is a biomarker produced in a large amount from malignant tumor cells, in real time, so that it may be determined whether a subject is afflicted with malignant tumor in a biological fluid by a non-invasive method.

In one specific exemplary embodiment, the cancer may be skin cancer, gastric cancer, liver cancer, lung cancer, colorectal cancer, uterine cancer, or breast cancer, but is not limited thereto.

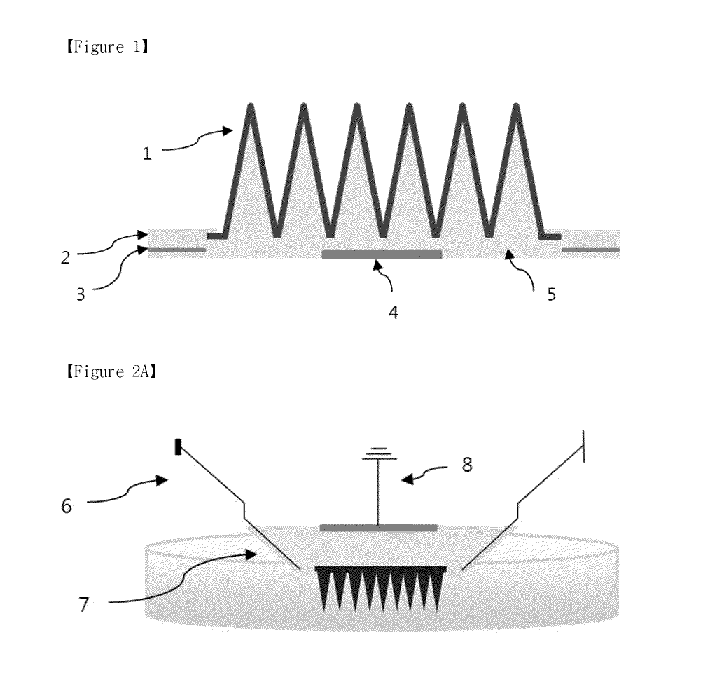

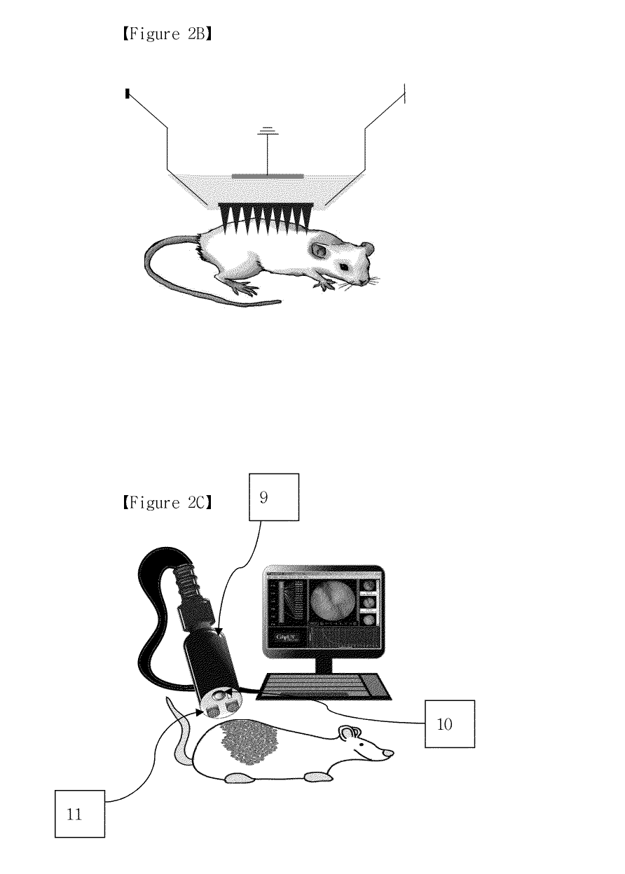

FIGS. 1 and 2 illustrate a sensor for detecting nitrogen monoxide, including the microneedle according to an exemplary embodiment of the present invention. FIG. 1 illustrates a sensor for detecting nitrogen monoxide, including the microneedle of the present invention and an electrode. FIG. 2 illustrates an exemplary aspect of a sensor which is configured for actually detecting nitrogen monoxide present in vitro and in vivo. The sensor for detecting nitrogen monoxide according to the present invention is an electrochemical sensor, and may measure current flowing in the channel composed of PEDOT and hemin by sequentially depositing titanium, chromium, and gold on both ends and a bottom portion of the microneedle to form a total of three electrodes, and allowing each of the electrodes to function as a reference electrode, a working electrode, and a counter electrode.

When nitrogen monoxide is captured by iron of hemin molecules in a channel in which the PEDOT and the hemin molecule form a pi-pi bond, the resistance of the PEDOT varies as the density of pi electrons of the hemin molecule forming a pi bond with PEDOT is changed.

More specifically, the trivalent iron ions of the hemin molecule tend to receive one electron and become stabilized because the 4s orbital of the iron ions is in an empty state. Therefore, the trivalent iron ions become more stable while being bonded to a number of pi electrons present in the polymer chains of PEDOT to form a pi-pi bond. The PEDOT polymer is present in a p-type doping state by a pi-pi bond with the hemi molecules, and an electron carrier, which provides conductivity to the PEDOT polymer, becomes a positive hole flowing along the chains. In this case, when nitrogen monoxide composed of radicals approaches the hemin molecule, the trivalent iron ions of the hemin molecule accept electrons of nitrogen monoxide which forms a stronger bond, the electron density of iron ions is partially packed from nitrogen monoxide and dispersed, and the electron density is also transferred to the polymer chains of PEDOT, thereby increasing the electron density of PEDOT. Accordingly, a positive hole, which is an electron carrier of the p-type doped PEDOT polymer chains, is bonded to an electron by an additional inflow of electrons, and the density of the electron carrier is reduced while the function as the electron carrier is offset, thereby increasing the resistance of the sensor channel. That is, the presence and absence and amount of nitrogen monoxide may be detected depending on the degree of reduction in resistance according to the change in current of the microneedle measured in real time.

In one specific exemplary embodiment, the height of the microneedle of the present invention may be 300 to 1,000 micrometers, 400 to 900 micrometers, 500 to 800 micrometers, and 550 to 750 micrometers. When the height of the microneedle is within the range, the microneedle does not touch the nerves and causes no pain to a subject while being applied to the subject, and may sufficiently pass through the skin in the subcutaneous layer and access the portion around cancer, which is exposed to the dermal layer.

The manufacturing of the sensor for detecting nitrogen monoxide according to the present invention may additionally include performing a waterproof treatment, except for the microneedle part, after depositing the electrode on the microneedle pad. In one specific exemplary embodiment, the waterproof treatment may be performed by coating the sensor with a polymer for waterproof treatment, such as a silicon-based polymer, a parylene-based polymer, a non-conductive plastic, or a hydrophobic polymer. The sensor may be subjected to waterproof treatment by being coated with the polymer for waterproof treatment except for the PEDOT coating region for detecting nitrogen monoxide. When the portion other than the channel of the microneedle sensor is covered with the polymer for waterproof treatment, a large noise may be reduced because the electrode portion other than the channel does not directly touch an aqueous solution containing nitrogen monoxide or the skin, and the detection efficiency may be improved because the sensor has a form of sensing nitrogen monoxide only sensed in the channel.

The silicon polymer includes sinylon, polyurethane, epoxy, polydimethylsiloxane, decamethyl cyclopentasiloxane, and the like, but is not limited thereto. The parylene-based polymer includes Parylene-A, Parylene-B, Parylene-C polymers, and the like, but is not limited thereto.

The microneedle or the sensor for detecting nitrogen monoxide according to the present invention may be safely applied to a living organism by using materials which are innocuous to the living organism. For example, the sensor of the present invention may also be applied as it is to the skin, and may also be used while being attached to an endoscope for diagnosing cancer.

Accordingly, the present invention also provides an endoscope including the microneedle or the sensor for detecting nitrogen monoxide. FIG. 2c illustrates an aspect of an endoscope to which the microneedle or the sensor for detecting nitrogen monoxide according to the present invention is mounted.

The microneedle of the present invention has a small size of 0.5 cm² or less, and may be manufactured along with the endoscope. The sensor for detecting nitrogen monoxide may be electrically connected and attached to an endoscope which is typically used for diagnosing cancer. As the method for attaching the sensor for detecting nitrogen monoxide to the endoscope, a typical method publicly known may be used without limitation.

For example, the sensor for detecting nitrogen monoxide may be applied in vivo as a hose-type or a capsule-type depending on the kind of endoscope, and when the sensor is introduced in vivo, the channel part of the microneedle is introduced in vivo while being surrounded by a protective film, and the amount of nitrogen monoxide may be detected by dissolving the protective film immediately before sensing a specific tissue, and then pricking the tissue with the needle.

The microneedle or the sensor for detecting nitrogen monoxide according to the present invention may replace an existing tissue examination, which is cumbersome and takes a long period of time when the in vivo tumor is diagnosed by an endoscope. The endoscope including the microneedle or the sensor for detecting nitrogen monoxide according to the present invention may determine a malignant tumor conveniently and at low costs by confirming a position presumed to be an in vivo tumor cell through the endoscope, and measuring the concentration of nitrogen monoxide around the confirmed tissue as a change in current flow in an aqueous solution state in real time. That is, it is possible to directly distinguish in vivo whether the tumor is malignant or benign by confirming the position suspected to be a tumor by a dye method, and then inserting the microneedle in vivo instead of collecting the tissue. Further, the prognosis of a tumor may be rapidly and easily forecast by forecasting the size and growth degree of a tumor according to the degree of reduction in resistance based on a change in current of the microneedle, which is measured in real time. Since the microneedle may be applied to various tumors, the microneedle of the present invention may be mounted to various kinds of endoscopes, and variously diagnose characteristics of the tumors.

In one specific exemplary embodiment, the endoscope includes a gastroscope, a bronchial endoscope, a colonofiberscope, an esophageal endoscope, a duodenum endoscope, a bladder endoscope, a celioscope, a thoracic cavity endoscope, or a cardiac endoscope, and the like, but is not limited thereto.

Hereinafter, the present invention will be described in detail with reference to the following Examples. However, the following Examples are only for exemplifying the present invention, and the content of the present invention is not limited by the following Examples.

### Preparation Example 1

### Manufacture of Dopamine-PEDOT-Hemin Molecule Channel

A PDMS mold having 10 needle patterns in both width and length directions was filled with 0.5 g of beads of polycaprolactone having a molecular weight of 8,000 kDa, and then, the beads were dissolved in an oven at 180° C. under vacuum conditions for approximately 3 hours. After the mold was removed from the oven, the microneedle base cooled at normal temperature was separated from the mold. Dopamine hydrochloride at a concentration of 2 mg·ml⁻¹ was dissolved in a tris buffer at a pH of 8.5 and a concentration of 1.0 mM. And then, the polycaprolactone microneedle base was put into the solution, the resulting solution was stirred at 37° C. for 24 hours, and the surface of the microneedle base was coated with dopamine. In order to prepare a PEDOT channel, a solution mixed with PEDOT:PSS 1000 was filtered by a 0.45 mm filter, and then a solution process was performed by covering the microneedle base doped with dopamine with the solution, and drying the microneedle base at 37° C. in an oven. The surface of the microneedle was entirely coated with poly(methyl methacrylate) (PMMA), and then, only the channel portion of the microneedle was exposed by using e-beam lithography. Next, hemin molecules dissolved in an organic solvent of dimethyl sulfoxide (DMSO) at a concentration of 1 mg/ml were placed on the PEDOT channel, and settled for 24 hours. The PEDOT channel was washed three times each with DMSO and an isopropyl alcohol solution to remove hemin molecules, which were not bonded, from the PEDOT channel, thereby manufacturing a dopamine-PEDOT-hemin molecule channel.

### Preparation Example 2

### Manufacture of Microneedle Sensor

In order to manufacture three electrodes, titanium, palladium, and gold were sequentially deposited on the microneedle. AZ4620 photoresist was spin-coated on the surface of the microneedle, the microneedle was baked at 65° C. in an oven for 20 minutes, and then only a square-shaped site on which an electrode was to be deposited was selectively etched. Next, titanium, palladium, and gold were deposited in 10 nm, 10 nm, and 50 nm, respectively, by using an e-beam evaporator. And then, an unnecessary photoresist was all etched by washing the microneedle with isopropyl alcohol and distilled water. Finally, in order to subject a portion except for the PEDOT channel for detection to water proof treatment, the microneedle except for a channel site (5×5 mm) coated with PEDOT was coated with PDMS, and then cured at 60° C. in an oven, thereby manufacturing a microneedle sensor (1×1 cm). The configuration of the manufactured microneedle sensor is illustrated in FIGS. 1 and 2.

FIG. 1 is a cross-sectional view of a microneedle sensor for detecting nitrogen monoxide, and dopamine and PEDOT were sequentially coated on the microneedle base formed of polycaprolactone, and titanium (Ti), chromium (Cr), and gold (Au) were deposited on both ends of the microneedle, which were used as a counter electrode and a working electrode, respectively. Titanium and silver (Ag) for connecting a reference electrode were deposited on the rear surface of the microneedle, which was used as the reference electrode.

FIG. 2A illustrates an example of the configurations of a sensor for detecting nitrogen monoxide, including the microneedle according to the present invention. The amount of nitrogen monoxide present in the aqueous solution may be measured by connecting a copper conducting wire to each electrode of the microneedle as illustrated in FIG. 1, and then coating the microneedle with PDMS to subject portions except for the channel of the microneedle to waterproof treatment.

FIG. 2B illustrates an example of the aspects in which a sensor including the microneedle is inserted into a mouse with induced skin cancer, and only a channel portion of the microneedle portion subjected to waterproof treatment is allowed to be inserted into the skin of the mouse.

FIG. 2C illustrates an aspect in which a sensor including the microneedle according to the present invention is applied to an endoscope.

### Preparation Example 3

### Preparation of Solution for Providing Nitrogen Monoxide

In order to detect nitrogen monoxide in vitro, diethylamine NONOate sodium salt was dissolved in 10 mM of a PBS buffer having a pH of 7.4, in which 10 mM of NaOH had been dissolved, and the resulting solution was used as a supply source of nitrogen monoxide. Before the NONOate sodium salt was dissolved, oxygen was completely removed by bubbling 10 mM of the PBS buffer solution having a pH of 7.4, in which 10 mM of NaOH had been dissolved, with nitrogen for 2 hours, and then the NONOate sodium salt was dissolved immediately before a nitrogen monoxide detection test was performed by the sensor, thereby providing nitrogen monoxide.

### Preparation Example 4

### Macrophage Cell Treatment which Releases Nitrogen Monoxide

After the RAW 264.7 macrophage cells were grown in a DMEM cell culture solution for about one day, the first group was a control and was not treated with any reagent, the second group was a group rich in nitrogen monoxide and was subjected to treatment of 0.5 ug/ml of lipopolysaccharide (LPS) with a cell culture solution in order to cause nitrogen monoxide to be produced in a large amount, and the third group was a group in which the amount of nitrogen monoxide had been reduced and was subjected to treatment with aminoguanidine at a concentration of 100 mM along with 0.5 ug/ml of lipopolysaccharide.

### Preparation Example 5

### Detection of Cancer Cell by Using Mouse with Induced Skin Cancer

In order to construct a skin cancer mouse model, B 16F10 cells with a density of 1×10⁷ were put into both sides on the back of an SKH-1 mouse. After 2 weeks, when the skin cancer cells with a size of 0.5 cm³ were sufficiently grown, the current was measured by thoroughly cleaning the skin on the cancer cells with ethanol, and inserting the microneedle into the skin.

### Experimental Example 1

### Test of Properties of Microneedle Sensor

1-1) Surface Test of Microneedle Sensor

In order to confirm whether up to the needle end portion of the microneedle manufactured in Preparation Example 1 had been coated well with the conductive PEDOT channel, the surface of the microneedle sensor was observed by an optical microscope and a scanning electron microscope. Furthermore, in order to observe the hemin molecules coated on the PEDOT channel, various signals emitted by interaction of the sample surface with electron beam were analyzed by using an EDX detector which may analyze the constituent elements and relative amount of a material, and it was confirmed whether hemin molecules were present or not by detecting characteristic X-ray to qualitatively analyze chemical components having a micro structure. Further, in order to confirm the iron composition and chemical bonding state of the hemin molecule on the surface of the sample by measuring the photoelectron energy emitted by allowing characteristic X-ray to be incident to the surface of the microneedle, XPS was performed.

1-2) Analysis Result

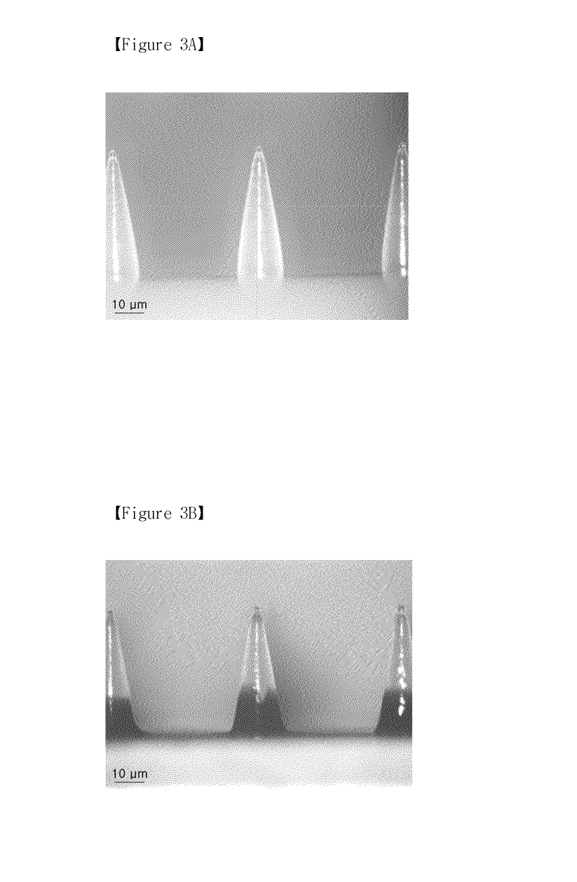

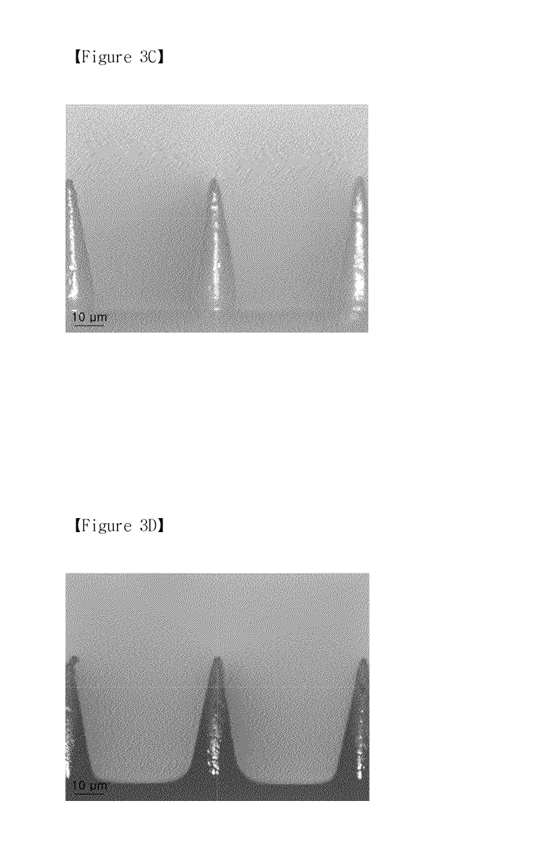

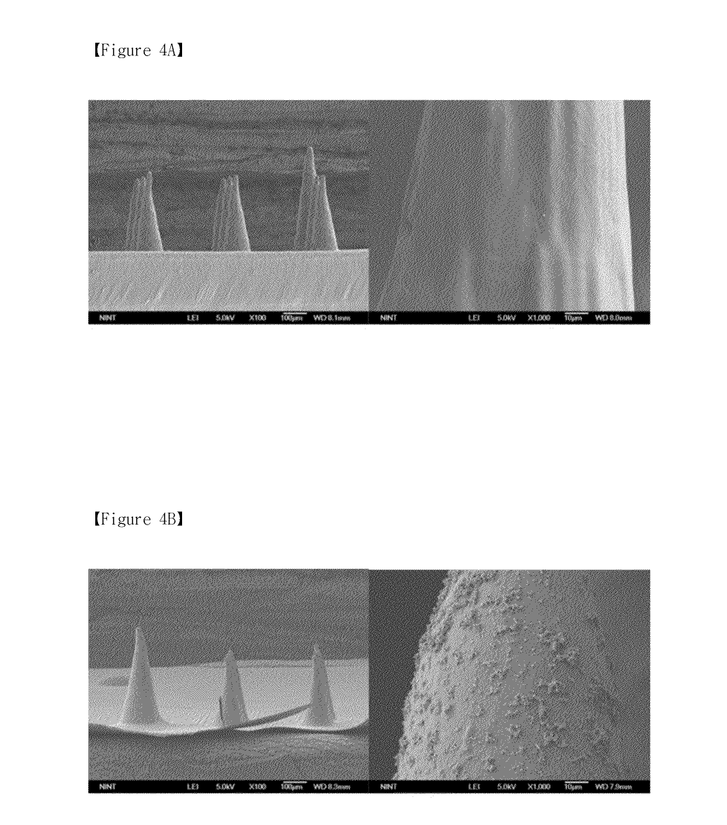

The result is illustrated in FIGS. 3 and 4. As a result of observation by an optical microscope, FIG. 3A illustrates a microneedle base composed of polycaprolactone. It can be seen that an imperfect coating was obtained when only the PEDOT was coated by subjecting the PEDOT to solution processing without dopamine coating (FIG. 3B), and it was confirmed that up to the needle end portion was coated well when polycaprolactone was coated with dopamine (FIG. 3C) and the PEDOT channel was coated with dopamine, and then was coated by subjecting the PEDOT to solution processing (FIG. 3D).

As a result of observation by a scanning electron microscope, it was confirmed that the surface of the initial microneedle (microneedle base) composed of polycaprolactone was clearly present (FIG. 4A). As a result of observation of the surface aspects of the microneedle coated with the PEDOT polymer without dopamine (FIG. 4B) and the microneedle coated with PEDOT after being coated with dopamine (FIG. 4C), it was confirmed that PEDOT was not peeled off from the polycaprolactone microneedle base and up to the end portion of the microneedle could be coated with PEDOT only when the microneedle was coated with dopamine which functions as adhesion.

Accordingly, it could be seen from the images obtained by the optical microscope and the scanning electron microscope that the PEDOT channel was not separated from the microneedle and up to the end portion of the needle was coated well only when the microneedle coated with dopamine was subjected to a solution process of the PEDOT channel.

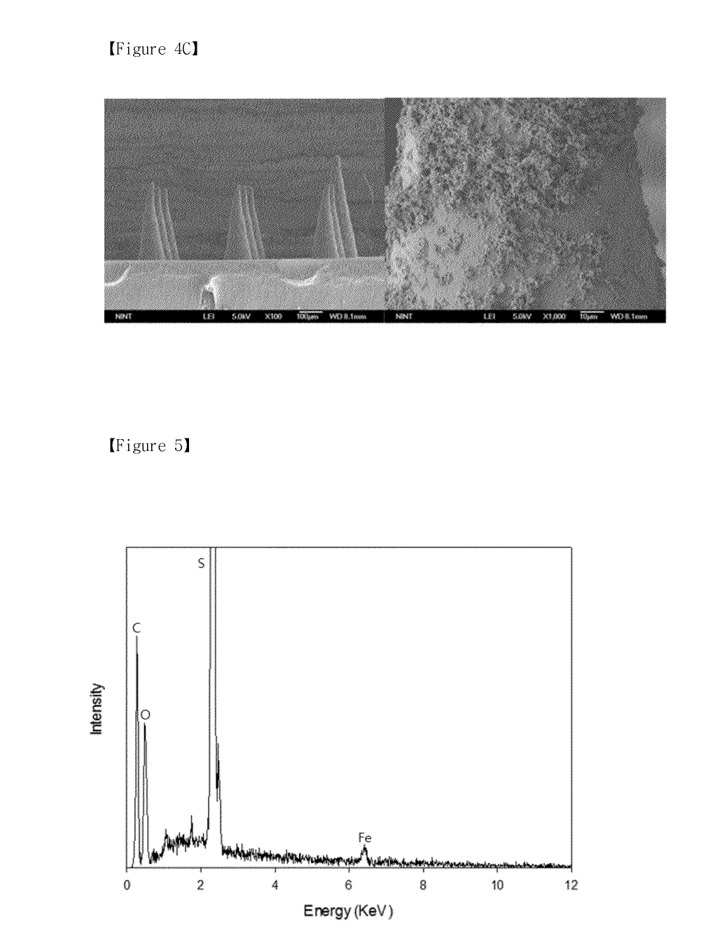

As illustrated in FIG. 5, a peak of iron at a very low intensity was observed as a result of measurement with a spot size 4 and an energy of 20 keV from the EDX graph. Since a peak can be obtained when only relative amounts of constituent elements are compared on the assumption that the constituent elements are bulk materials during the EDX measurement and 0.1% or more of the constituent element is contained, from the fact that an inherent peak of only iron was produced at a position around 6.4 eV, it could be seen that iron was present at a ratio lower than carbon, oxygen, sulfur, and sodium which are components of the PEDOT channel, but a certain sufficient amount of iron was present on the surface.

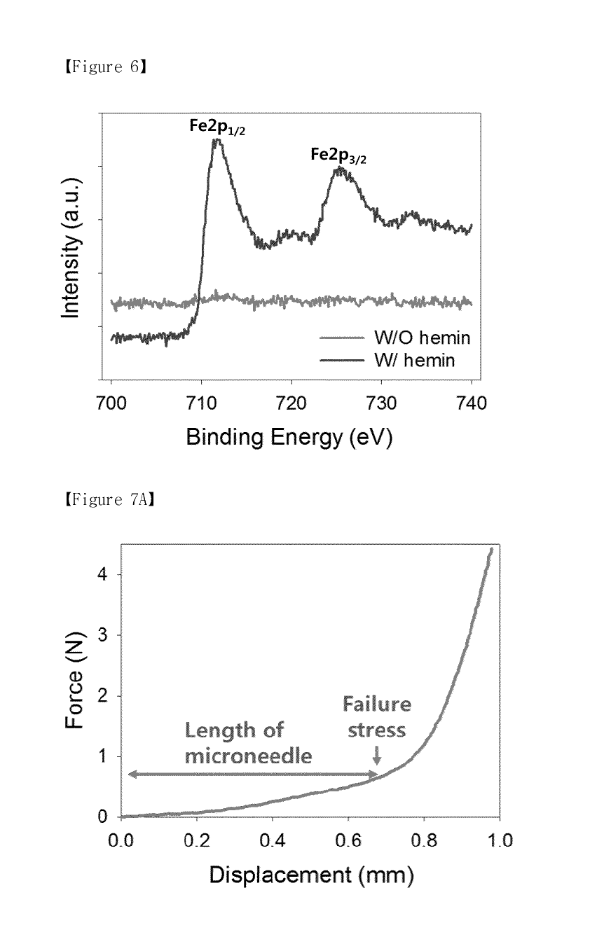

Further, as illustrated in FIG. 6, it was confirmed that iron was present on the microneedle channel from the iron analysis graph using XPS. This means that a hemin group was stably bonded on the PEDOT. Since a peak of Fe2p, which means a non-covalent bond with the PEDOT molecule, was observed and a high value of Fe2p_{3/2} was obtained at an energy around 710 eV in the XPS, it could be seen that hemin molecules were maintained as one layer on the PEDOT channel.

### Experimental Example 2

### Mechanical Properties of Microneedle

1-1) Measurement of Strength of Microneedle

In order to confirm mechanical properties of the microneedle, pressure (stress) was measured according to the measurement of strength, failure stress, and displacement length (strain). A graph in change of forces was obtained at a speed of 0.5 mm/min according to the compression length by using an Instron eXpert 760 mechanical tester (ADMET), and a compression constant was obtained by converting the graph into a graph related to stress and strain. In addition, holes produced on the skin by substantially inserting the microneedle into the skin of the mouse were observed by an optical microscope.

1-2) Analysis Result

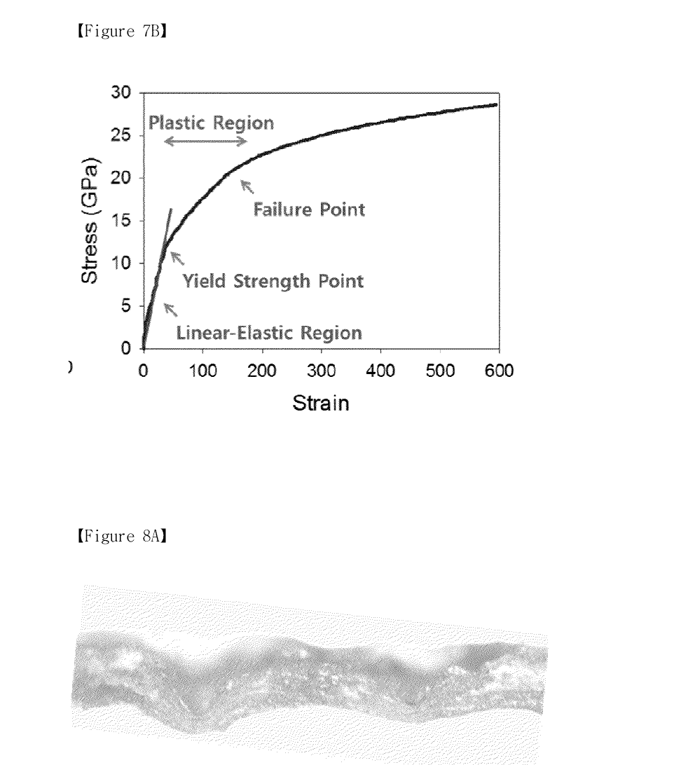

As illustrated in FIG. 7A, as a result of measuring the strength of the microneedle, it could be seen from the graph of forces according to the compression length that the failure stress shown at a position of 0.7 mm, which was a needle length, was about 1.20±0.11 N. A failure stress of 1 N or more means that the microneedle may be inserted into the skin without defects.

Furthermore, as illustrated in FIG. 7B, it was confirmed that the compression stress was about 162.5±0.31 MPa, and mechanically strong properties were obtained as an elastic region shown as a straight line in the early stage is obtained from the stress and strain graph.

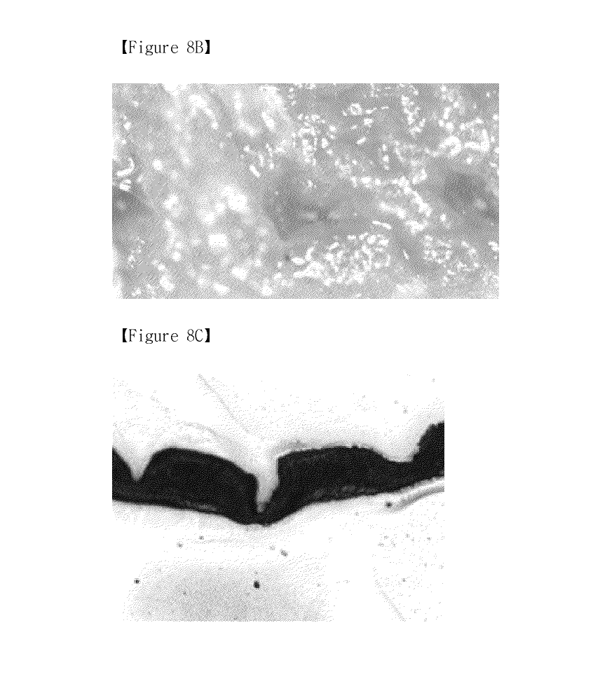

As illustrated in FIG. 8, it was confirmed through the analysis of the side view (FIG. 8A), the cross-sectional view (FIG. 8B), and the cryosection (FIG. 8C) of the skin of the mouse into which the microneedle was inserted that the microneedles were uniformly pricked on the skin of a mouse at regular intervals.

### Experimental Example 3

### Surface Properties of Microneedle Sensor Using Circulating Current Method

1-1) Measurement of Current According to Voltage Using Circulating Current Method

For the surface analysis of the sensor, the circulating current was measured at each coating step of dopamine, PEDOT, and hemin molecules by using a CHI 832 workstation (Shanghai Chenhua, China). As an electrolyte, a PBS buffer solution with a pH of 7.4 was selected, and the current measurement was performed at a scan rate of 50 mV/s. A graph of measuring the circulating current of the microneedle sensor was obtained according to the scan rate.

Further, an oxidation and reduction peak of nitrogen monoxide was analyzed by treating the PBS buffer solution with nitrogen monoxide at each concentration, and measuring the circulating current.

In order to measure the resistance of the surface of the microneedle sensor, electrochemical impedance spectra (EIS) were measured by using a CHI 660 electrochemical workstation. The impedance spectra were measured in the PBS buffer solution with a pH of 7.4 under an ac of 5 mV according to the frequency in the range from 0.1 Hz to 100 KHz.

1-2) Analysis Result

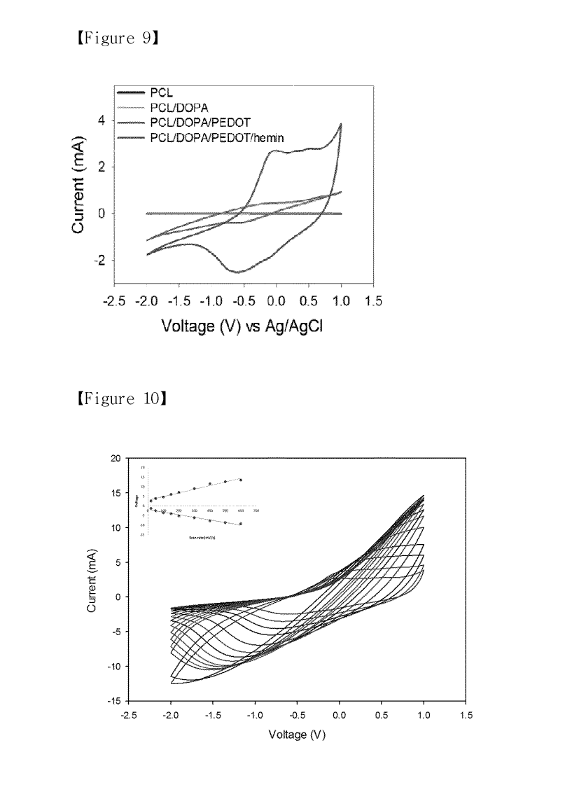

As illustrated in FIG. 9, it could be seen from the voltage-current graph according to the measurement of the circulating current that in the microneedle composed of polycaprolactone and the needle coated with polycaprolactone and dopamine, current rarely flowed in a voltage range from −2.0 V to 1.0 V. In contrast, a small amount of change in current was observed in the conductive polymer. In the case of the needle coated with a hemin group, the current flow was increased by hemin molecules, which transport electrons well, and an inherent oxidation-reduction peak of a hemin group alone could be observed. The oxidation-reduction peak of a hemin group alone was observed at −0.4 V and −0.6 V, respectively. The oxidation-reduction peak of a hemin group alone means that the oxidation state had been reversibly changed from trivalent iron ion to divalent iron ion.

As illustrated in FIG. 10, the voltage absolute value of the peak in which the oxidation-reduction occurred was increased as the scan rate was increased, and the current value in the peak was also increased, and the entire current values of the graph were also increased. This means that electrochemical characteristics of the sensor were exhibited well. The graph inserted into the graph of FIG. 10 is a graph illustrating the voltage at which the oxidation-reduction occurred according to the scan rate, and means from the fact that both the voltage to be oxidized and the voltage to be reduced had a tendency as a linear function that the surface of the electrochemical sensor exhibits an electrochemical tendency well.

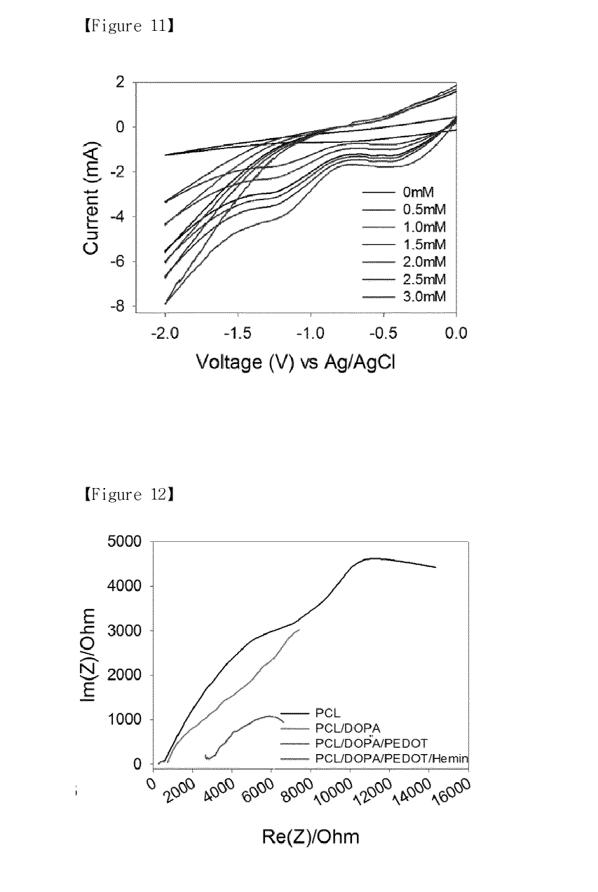

As illustrated in FIG. 11, only an oxidation-reduction peak of a hemin group was observed in a nitrogen monoxide-free environment, but when nitrogen monoxide was added to the electrolyte, a reduction peak of nitrogen monoxide was also observed at −1.10 V. Further, it could be seen that the higher the concentration of nitrogen monoxide was, the higher the value of current flowing was. Since the hemin molecules bonded to the PEDOT channel selectively capture nitrogen monoxide, the amount of current flowing in the sensor is increased.

As illustrated in FIG. 12, the resistance value of the microneedle was 450Ω, the resistance value of the microneedle with only a PEDOT channel was 6,000Ω, and the surface resistance of the microneedle sensor coated with was the lowest. This means that the hemin group plays a great role in transporting electrons in the electrolyte during the EIS measurement.

### Experimental Example 4

### Microneedle Stability Test

1-1) Observation of Surface of PEDOT Channel Before and after Inserting Microneedle into Skin

The channel surface of the microneedle was observed through a scanning electron microscope before and after the microneedle was inserted into the skin of the mouse. Further, it was confirmed through the confirmation of the oxidation-reduction peak of hemin molecules by measuring the circulating current under the same conditions as in Experimental Example 3 after washing the microneedle sensor with a DMSO organic solvent for 5 days that hemin molecules could be bonded well to the PEDOT channel, and a change in graph was observed by measuring the circulating current 50 consecutive times under the same conditions as described above.

1-2) Analysis Result

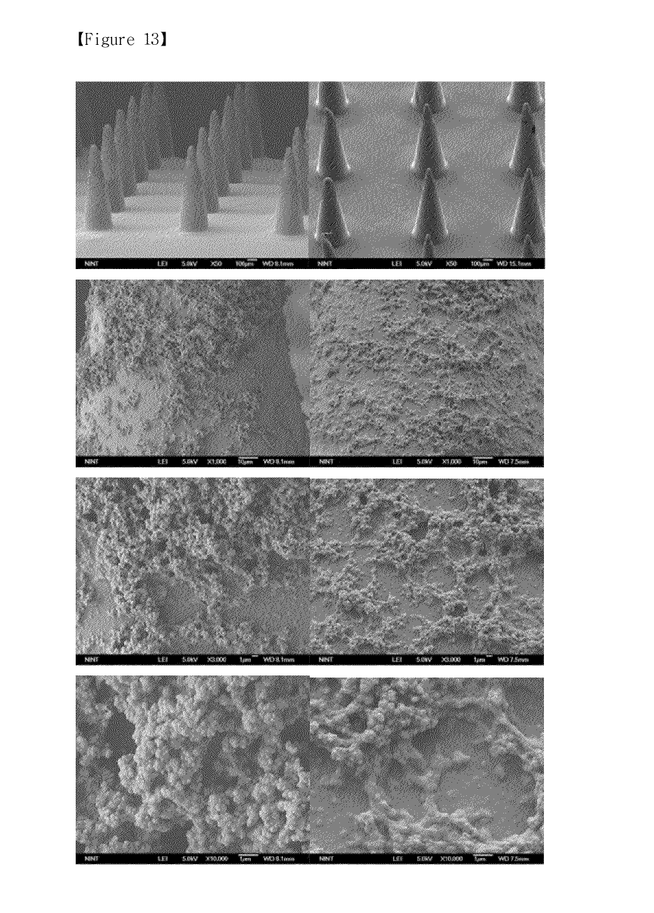

As a result of observing the surface state of the microneedle before and after the microneedle was inserted into the skin by a scanning electron microscope, it could be confirmed as illustrated in FIG. 13 that there was no great change in the surface, and the PEDOT polymer was bonded well. That is, the PEDOT coating layer was not greatly damaged before and after the microneedle was inserted.

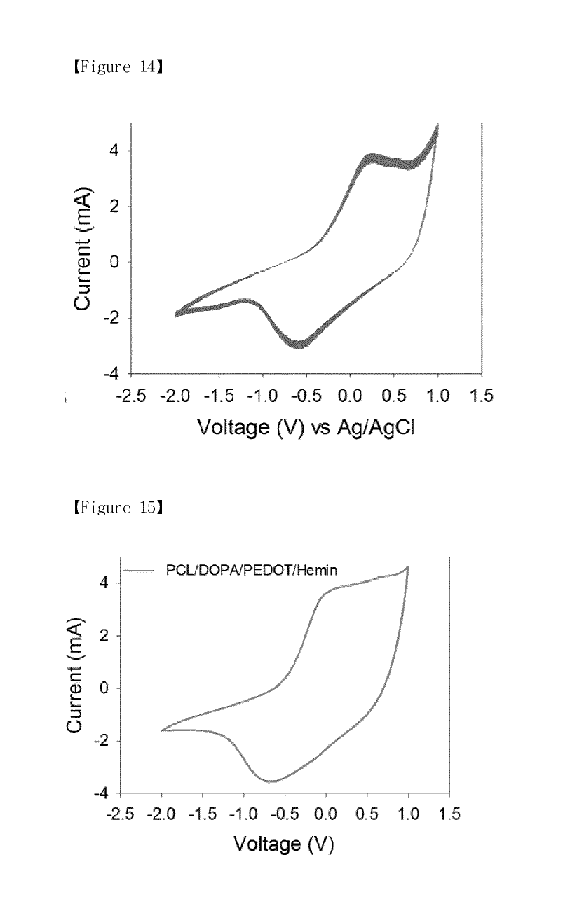

As illustrated in FIG. 14, the oxidation-reduction peak of a hemin group was exhibited in the same position as in Experimental Example 3 even after the microneedle sensor was washed with an organic solvent for 5 days even in the circulating current graph, and as illustrated in FIG. 15, it could be seen that only a difference by about 2.7% was exhibited even in the circulating current graph subjected to 50 times of the cycle when the change in current at the 50th time was compared with the change in current at the first time. The result means that the microneedle sensor is in a very stable state in the aqueous solution.

1-3) Quantification of Hemin Molecules Bonded to PEDOT Channel

The number of moles of hemin molecules attached to the microneedle was calculated. First, a standard of the concentrations of hemin molecules was obtained according to the intensity of absorbance at 405 nm by obtaining the UV spectra at each concentration of hemin molecules dissolved in DMSO. Next, the microneedle coated with the PEDOT channel was immersed in 0.1 ml of a solution of hemin molecules at an initial concentration of 1 mg/ml for one day, and then the concentration of hemin molecules left in the solution was calculated by using the UV spectrum to measure the intensity of absorbance.

1-4) Analysis Result

The concentration of hemin molecules was calculated according to the following equations by using the UV spectrum.

Sₕₑₘᵢₙ−Nₕₑₘᵢₙ/S_{pEDOT}

Nₕₑₘᵢₙ=Nₕₑₘᵢₙ₀−Nₕₑₘᵢₙ₁=(Cₕₑₘᵢₙ₀−Cₕₑₘᵢₙ₁)×V

In this case, Sₕₑₘᵢₙ means a concentration of hemin molecules bonded on the PEDOT channel per surface area, Nₕₑₘᵢₙ means the total number of hemin molecules bonded to the PEDOT channel, Nₕₑₘᵢₙ₀ means the number of initial hemin molecules in the solution before the microneedle is loaded, and Nₕₑₘᵢₙ₁ means the number of hemin molecules left in the solution after the microneedle is loaded. The difference between Nₕₑₘᵢₙ₀ and Nₕₑₘᵢₙ₁ indicates the number of hemin molecules bonded to the microneedle. S_{PEDOT} is an area of a PEDOT channel and 1 cm², Cₕₑₘᵢₙ₀ is 1 mg/ml which is an initial concentration of a hemin group, and Cₕₑₘᵢₙ₁ is a concentration of hemin molecules obtained from the UV spectrum. Since Sₕₑₘᵢₙ=0.91 nm⁻² through the equations, it could be seen that the concentration of hemin molecules was a concentration at which the hemin molecules are bonded on the PEDOT channel as a monolayer, considering that the diameter of hemin molecules is 0.5 nm.

### Experimental Example 5

### Cytotoxicity Test of Microneedle

### 1-1) 3-(4,5-Dimethylthiazol-2-yl)-2, 5-diphenyltetrazolium bromide, MTT Test

In order to measure the degree of cytotoxicity of the microneedle coating layers, the MTT reagent, which measures the activity of cells, was used. The Raw 146.7 macrophage cells were grown in the same number each on the polycaprolactone microneedle, the microneedle coated with dopamine, the microneedle coated with both dopamine and the PEDOT channel, and the microneedle coated with even dopamine, the PEDOT channel, and a hemin group, the cells were completely attached thereto after 1 to 2 days, and then 150 ml of the MTT solution filtered by a 0.2 mm filter at a concentration of 0.5 mg ml⁻¹ was administered to the cells. The needles were warmed at 37° C. in an oven for about 2 hours, and the solution on the needle was completely removed. And then, 200 μl of DMSO was put into the microneedles to completely dissolve cells which turned purple, and then the absorbance was measured in a wavelength range of 540 nm by using the ELISA microplate reader (molecular devices, Versamax).

### 1-2) Analysis Result

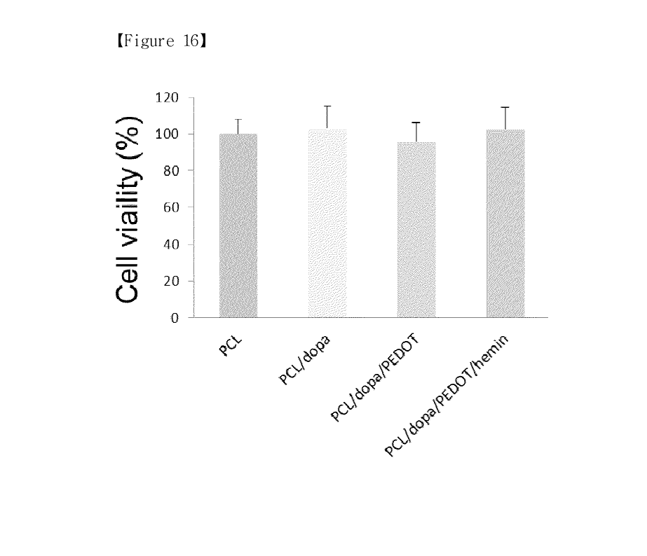

As illustrated in FIG. 16, when the cell viability graph was observed, the stability of polycaprolactone and dopamine was approximately 100% and 103.2%, respectively, meaning that cells almost survived, and it was confirmed that the cell viability on the PEDOT channel was 95.6% and the cell viability on the microneedle coated with up to hemin molecules was 102.3%, meaning that most cells survived well on the microneedles. This means that the microneedle sensor had almost no cytotoxicity.

### Experimental Example 6

### In Vitro Nitrogen Monoxide Detection Test

1-1) Real-Time In Vitro Nitrogen Monoxide Detection Test at Each Concentration

As illustrated in FIG. 2, a microneedle which entirely covered portions, except for the channel portion, with PDMS was used. In the channel surrounded with PDMS, two holes were perforated in the PDMS chamber, and then, were connected to an injection needle to allow the solution to flow into one hole and to be escaped out of the other hole. According to Preparation Example 3, nitrogen monoxide solutions were prepared at a concentration of 1, 2, 4, 8, and 16 mM, and then, were flowed into the channel portion of the microneedle by using a syringe. For the measurement of current flowing in the microneedle, the evaporation of the solution was minimized by using a chamber-type probe station (Lake Shore Probe Station (Model TPP4)), three probe styluses were connected to three electrodes of the microneedle sensor, and then the value of flowing current was measured by applying 0.1 V to a working electrode and a counter electrode, and applying 0.07 V between the counter electrode and a reference electrode. The graph was obtained by performing the experiment on a microneedle sensor that is bonded to a hemin group, and a microneedle sensor that is not bonded to a hemin group and has only PEDOT.

1-2) Analysis Result

As illustrated in FIG. 17, the current value in an equilibrium state was 49.9 μA while a solution free from nitrogen monoxide was placed on the channel of the microneedle sensor, and for a microneedle sensor (W/O hemin) which was not bonded to a hemin group, the value was still maintained even though a nitrogen monoxide solution each prepared at a concentration of 1, 2, 4, 8, and 16 mM was flowed. In contrast, it could be seen that when a nitrogen monoxide solution each prepared at a concentration of 1, 2, 4, 8, and 16 mM was injected into the microneedle sensor (W/hemin) bonded to hemin molecules, the equilibrium current value was sequentially lowered to 49.01, 47.61, 45.31, 41.62, and 33.89 uA starting from an equilibrium current value of 49.98 in proportion to the concentration of nitrogen monoxide, and the current was decreased by 0.97, 1.40, 2.30, 3.69, and 7.73 uA. This means that the higher the concentration of nitrogen monoxide having a radical form having extra electrons is, the larger amount of electrons hemin molecules bonded to the microneedle accept, and accordingly, the number of positive holes, which are an electron carrier of the PEDOT channel doped with a p-type dopant, is decreased, thereby reducing conductivity. That is, it could be seen that the microneedle sensor according to the present invention selectively detected nitrogen monoxide well.

### Experimental Example 7

### Selectivity Test of Microneedle Sensor

1-1) Measurement of Reaction to In Vivo Various Polysaccharides and Proteins by Microneedle

Before the microneedle was substantially applied in vivo, it was examined whether the conductivity of the microneedle channel was changed by various kinds of polysaccharides and proteins with a high electron density present in vivo. Solutions in which galactose, glucose, a trivalent iron ion, peroxide, an ovalbumin protein, lysozyme, and a bovine serum albumin protein are dissolved at a concentration of about 1 uM were sequentially flowed into the microneedle sensor, and finally, nitrogen monoxide at 1 and 2 uM was sequentially flowed into the microneedle sensor.

1-2) Analysis Result

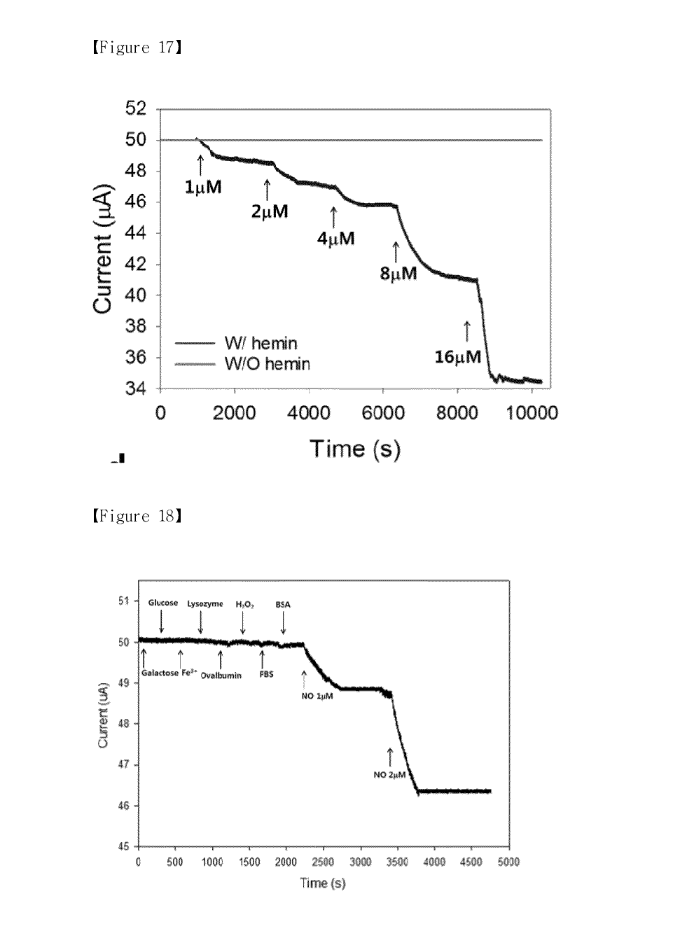

As illustrated in FIG. 18, it was confirmed that a change in conductivity of the microneedle sensor was not observed by galactose, glucose, a trivalent iron ion, peroxide, an ovalbumin protein, lysozyme, and a bovine serum albumin protein, and 50.3 uA, which is an initial equilibrium current of the microneedle, was continuously maintained. □□However, when nitrogen monoxide at 1 and 2 uM was flowed, it could be seen that the current was reduced by 1.01 uA and 1.97 uA, respectively, from the initial current value, and the conductivity of the microneedle was reduced. This result means that the microneedle sensor bonded to hemin may selectively detect nitrogen monoxide.

1-3) Real-Time Detection Test of Nitrogen Monoxide in Cell Culture Solution (Dulbecco's Modified Eagle Medium (DMEM))

A real-time detection test of nitrogen monoxide was performed in a cell culture solution including various proteins, amino acids, vitamins, and inorganic salts by replacing the PBS solution with DMEM in the same manner as in the real-time detection test of nitrogen monoxide performed in Experimental Example 6.

1-4) Analysis Result

As illustrated in FIG. 19, a slightly unstable change in current was exhibited, but when a nitrogen monoxide solution prepared at a concentration of 1, 2, 4, 8, and 16 uM was flowed, the current value was reduced to 49.47, 46.99, 41.04, 30.01, and 15.13 uA starting from a current value of 51.19, which flowed in an initial equilibrium state of the microneedle, similarly to the result of Experimental Example 6. That is, it could be seen that the current was reduced by about 1.72, 2.48, 5.95, 11.03, and 14.88 uA at each step in proportion to the concentration of nitrogen monoxide flowed.

### Experimental Example 8

### Grease Reagent Test

1-1) Conversion of Concentration of Nitrogen Monoxide

In order to examine the substantial amount of nitrogen monoxide emitted from the three groups of the macrophage cells of Preparation Example 4, the absorbance of the solution in a wavelength range of 540 nm was obtained depending on the concentration of nitrogen monoxide emitted from the quantified diethylamine NONOate sodium salt by using a grease reagent test. The absorbance was measured at a total of 11 concentrations by continuously diluting the amount of nitrogen monoxide emitted by using diethylamine NONOate sodium salt by ½ from 250 uM. 100 ul of the solution of diethylamine NONOate sodium salt at various concentrations was mixed with 100 ul of a grease reagent, and 10 minutes later, a standard was obtained based on the intensity value of the absorbance in a wavelength range of 540 nm.

1-2) Analysis Result

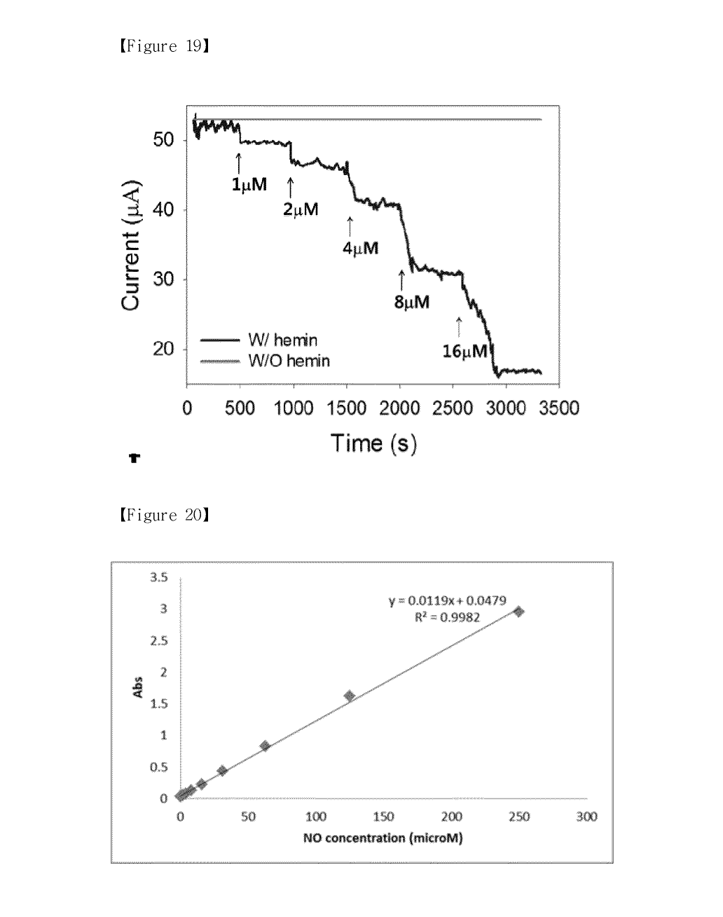

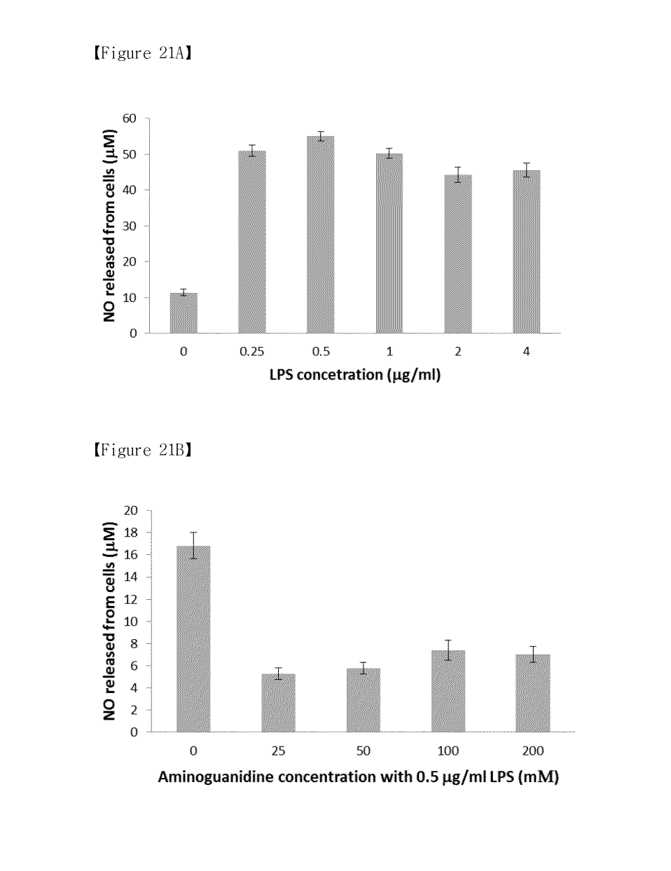

As illustrated in FIG. 20, the absorbance depending on various concentrations of the NONOate sodium salt was obtained, and the relationship of y=0.0119x+0.0479 was obtained therefrom. In order to quantify the amount of nitrogen monoxide released from cells in the following Experimental Example 9, the relationship equation was used.

### Experimental Example 9

### Nitrogen Monoxide Detection Test of Cells

1-1) Real-Time Detection of Nitrogen Monoxide Generated from Macrophage Cells

During the metabolism process of the living RAW 264.7 macrophage cells, a maximum nano molarity of nitrogen monoxide was generated. When a cell medium which grows the macrophage was treated with a lipopolysaccharide, nitrogen monoxide at a concentration which is 1,000 times or higher than a usual concentration by increasing the differentiation of an inducible nitric oxide synthase (iNOS) which is one of the enzymes which produce nitrogen monoxide from cells. Further, when the cell medium was treated with aminoguanidine along with the lipopolysaccharide, the lipopolysaccharide was suppressed to again reduce the amount of nitrogen monoxide emitted from the cells.

As illustrated in FIG. 2A, about 10,000 cells were cultured on a microneedle, in which the portion other than the channel was molded well with PDMS, for about one day, were divided into three groups, among which a group treated with nothing, a group treated with 0.5 ug/ml of a lipopolysaccharide, and a group treated with 0.5 ug/ml of the lipopolysaccharide and 100 mM of aminoguanidine were prepared, and activated at 37° C. in an incubator for 30 hours to emit sufficient nitrogen monoxide for 36 hours. And then, a copper conducting wire connected to the microneedle sensor was connected to the probe station to perform an experiment in the same manner as in the current measurement method performed in Experimental Example 6. The experiment was performed likewise on the microneedle to which hemin molecules were not bonded.

1-2) Quantification of Amounts of Nitrogen Monoxide Generated from Three Groups of Cells

In order to analyze the production degree of nitrogen monoxide from the macrophage according to the concentrations of lipopolysaccharide (LPS) and aminoguanidine, a grease reagent test was performed. 10,000 macrophages were cultured in a 6-well plate for cell culture for one day, and then treated with lipopolysaccharide at 0, 0.25, 0.5, 1, 2, and 4 μg/ml, a grease test was performed one day later, and the absorbance produced from the UV spectrum was converted by using the nitrogen monoxide concentration relationship according to the absorbance obtained in FIG. 20, and illustrated in FIG. 21A. In addition, the group treated with lipopolysaccharide at 0.5 ug/ml was treated with aminoguanidine at 25, 50, 100, 200, and 400 mM, respectively, a grease test was performed one day later by the same method, and then the amount of nitrogen monoxide generated from each macrophage cell group was converted and illustrated in FIG. 21B.

1-3) Analysis Result

As a result, as illustrated in FIG. 21A, it could be seen that nitrogen monoxide was generated from the macrophage cells according to the treatment with lipopolysaccharide. When the cells were treated with LPS at 0.5 um/ml, nitrogen monoxide was generated most efficiently. As illustrated in FIG. 21A, it could be seen that when the cells were treated with LPS along with aminoguanidine, LPS was suppressed by aminoguanidine, and thus, nitrogen monoxide was suppressed from being generated.

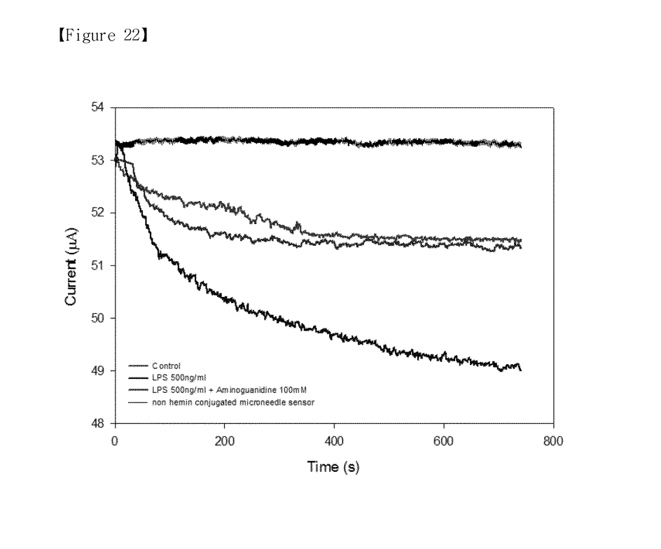

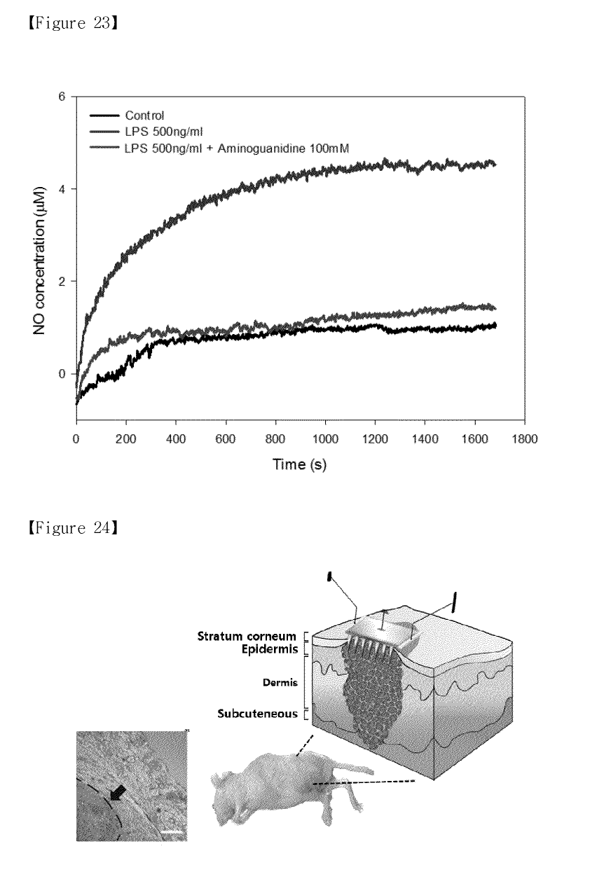

As illustrated in FIG. 22, in the case of a microneedle sensor free from hemin molecules, nitrogen monoxide from the macrophages was not detected while the initial microneedle equilibrium current value was continuously maintained. The microneedle sensor bonded to hemin molecules could detect nitrogen monoxide emitted from the macrophage cells in real time. Current was reduced by about 0.97 uA for the group which had not been treated with the reagent, 4.93 uA for the group which had been treated with lipopolysaccharide, and 1.03 uA for the group which had been treated with lipopolysaccharide and aminoguanidine. This can be seen as a decrease in conductivity of the microneedle channel by nitrogen monoxide emitted from the macrophage. The amount of nitrogen monoxide emitted, which was converted from the amount of reduction in current was calculated as 0.97, 4.93, and 1.03 uM, respectively, and it could be confirmed that these values were very similar to 0.96, 5.57, and 0.68 uM, which were the amounts of nitrogen monoxide emitted from the macrophages, which were converted through the grease reagent test performed in 1-2 (FIG. 23).

### Experimental Example 10

### In Vivo Nitrogen Monoxide Detection Test

1-1) Real-Time Measurement of Nitrogen Monoxide by Using Mouse with Induced Skin Cancer

The microneedle which was bonded to hemin molecules, and the microneedle which was not bonded to hemin molecules were pricked on the site on which the skin cancer cells were grown, thereby measuring the real-time current change. As a control, a general mouse without induced skin cancer was used.

1-2) Analysis Result

FIG. 24 illustrates that the skin tissue of a mouse with skin cancer is observed, and that cancer is detected by applying the sensor including the microneedle to the skin tissue of the mouse.

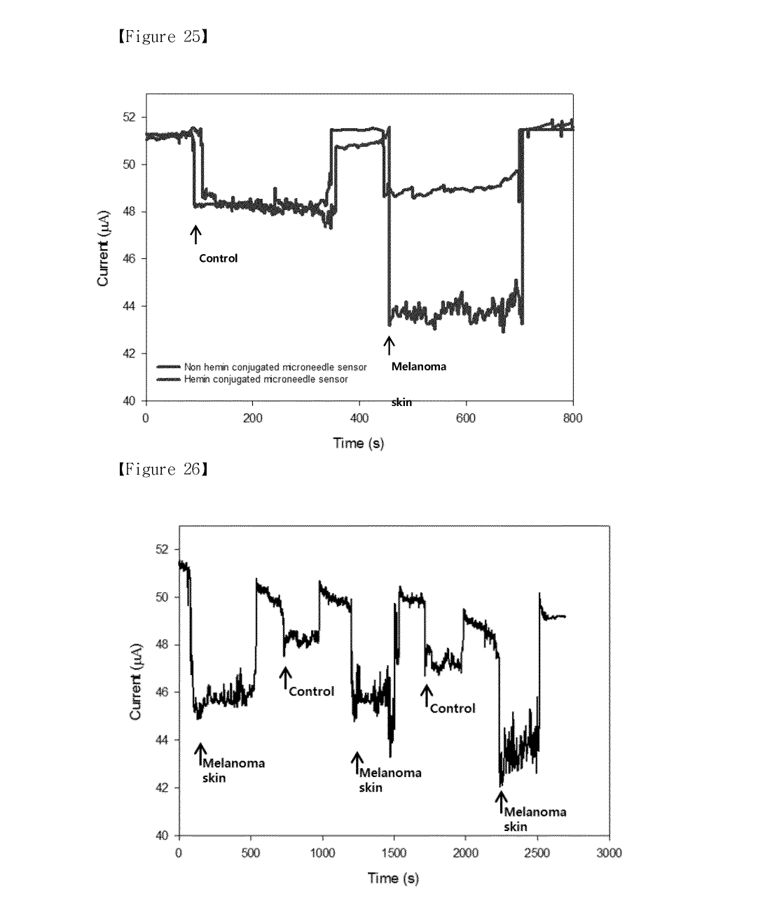

As illustrated in FIG. 25, in the case of the microneedle sensor connected to hemin molecules, a decrease in current occurred by about 2.92 uA compared to the case in which the microneedle sensor was inserted into the skin of a general mouse without induced skin cancer. And then, the equilibrium was reached again, and then when the microneedle sensor was inserted into the skin of the mouse with skin cancer, a decrease in current by 7.98 uA was exhibited. When a microneedle sensor composed of only PEDOT without hemin molecules was inserted into a general mouse and a mouse with induced skin cancer, respectively, the microneedle sensor exhibited a decrease in current of 2.87 mA and 2.57 mA, which are similar values. The change in resistance of the microneedle without hemin molecules is at the same level as the change in resistance occurring when the microneedle bonded to hemin molecules was inserted into the general mouse, and it can be seen that the value of change in current occurring when the microneedle is inserted in the general mouse is only an increase in resistance due to contact with the skin. That is, a decrease (t=85 s) in current occurring when the microneedle is inserted into a general mouse may be considered as an increase in contact resistance by inserting the microneedle channel into the skin. Therefore, a net current change value, which was changed by inserting the microneedle sensor into the skin cancer cells to detect nitrogen monoxide present in the tissues around the skin cancer, could be presumed to be 7.98 uA−2.92 uA=5.06 uA except for the value of increase in current due to contact with the skin.

FIG. 26 illustrates the results of measuring the decrease in current by inserting the microneedle sensor, which is repeatedly connected to hemin molecules, into the skin of a general mouse and the skin of a mouse with induced skin cancer cells. Likewise, it could be confirmed that when the microneedle sensor was inserted into a mouse with induced skin cancer, a decrease in current occurred two or more times higher than an increase in resistance by contact, which is generated when the microneedle sensor was inserted into a general mouse. This result means that the microneedle sensor of the present invention may sufficiently detect nitrogen monoxide repeatedly emitted in a large amount around the cancer cells in real time.

What is claimed is:

1. A microneedle in which a microneedle base; an adhesive polymer layer; a conductive polymer layer; and a nitrogen monoxide bonding molecule layer comprising iron ions are sequentially stacked.

2. The microneedle of claim 1, wherein the microneedle base is one or more selected from the group consisting of polyvinyl alcohol, polyethylene glycol, polylactide, polyglycolide, polyethylene oxide, polydioxanone, polyphosphazene, polyanhydride, polyamino acid, polyacrylate, polyacrylamide, polyurethane, polysiloxane, polyvinylpyrrolidone, polycaprolactone, polymethylmethacrylate, polyethylene, polyamide, polydimethylsiloxanes, polyester, polyorthoester, polycyanoacrylates, polyphosphazenes, polyvinylchrolide, polymethylpentene, polynitrobenzyl, polyaminoester, cellulose acetate butyrate, cellulose triacetate, polyethylene terephthalate, Teflon (polytetrafluoroethylene), stainless steel, silicon, silicon oxide, aluminum, aluminum oxide, nickel oxide, and SU-8.

3. The microneedle of claim 1, wherein the adhesive polymer is one or more selected from the group consisting of chitosan, silk, collagen, fibronectin, vitronectin, rubber, and polydopamine.

4. The microneedle of claim 1, wherein the conductive polymer is one or more selected from the group consisting of polyacetylene, polyaniline, polypyrrole, polythiophene, poly(1,4-phenylenevinylene), poly(1,4-phenylene sulfide), poly(fluorenylene ethynylene), polyisothianaphthene, polythienylene vinylene, polyphenylene vinylene, polyphenylene sulfide, polyhexylthiophene, PEDOT, and derivatives thereof.

5. The microneedle of claim 1, wherein a nitrogen monoxide bonding molecule comprising iron ions is a porphyrin ring or a hemin molecule, which has pi electrons in the core thereof.

6. A sensor for detecting nitrogen monoxide, comprising:
the microneedle of claim 1; and
an electrode.

7. The sensor of claim 6, wherein the electrode is one or more selected from the group consisting of nickel, chromium, titanium, gold, silver, and platinum.

8. The sensor of claim 6, wherein the electrode comprises a reference electrode, a working electrode, and a counter electrode.

9. A sensor for diagnosing cancer, comprising:
the microneedle of claim 1; and
an electrode.

10. The sensor of claim 9, wherein the cancer is skin cancer, gastric cancer, liver cancer, lung cancer, colorectal cancer, uterine cancer, or breast cancer.

11. An endoscope comprising: the microneedle of claim 1, the sensor for detecting nitrogen monoxide of claim 6, or the sensor for diagnosing cancer of claim 9.

12. The endoscope of claim 11, wherein the endoscope is a gastroscope, a bronchial endoscope, a colonofiberscope, an esophageal endoscope, a duodenum endoscope, a bladder endoscope, a celioscope, a thoracic cavity endoscope, or a cardiac endoscope.

13. A method for manufacturing a microneedle, the method including:
forming an adhesive polymer layer on a microneedle base by mixing the microneedle base with an adhesive polymer;
forming a conductive polymer layer on the adhesive polymer layer through a solution process by bringing the adhesive polymer layer into contact with a conductive polymer solution; and
forming a nitrogen monoxide bonding layer on the conductive polymer layer by bringing the conductive polymer layer into contact with a nitrogen monoxide bonding molecule layer comprising iron ions.

14. The method of claim 13, further comprising:
subjecting the microneedle base to UV treatment or ozone plasma treatment before forming the adhesive polymer layer on the microneedle base.

15. The method of claim 13, wherein the solution process is performed by immersing a microneedle base on which an adhesive polymer layer is formed in a conductive polymer solution, and drying the microneedle base.

16. A method for manufacturing a sensor for detecting nitrogen monoxide, the method comprising:
depositing an electrode on a microneedle pad in which the microneedle of claim 13 is formed.

17. The method of claim 16, further comprising:
performing a waterproof treatment, except for the microneedle part.

18. The method of claim 17, wherein the waterproof treatment is performed by coating the sensor with one or more selected from the group consisting of a silicon-based polymer, a parylene-based polymer, a non-conductive plastic, or a hydrophobic polymer.
